(19) United States
(12) Patent Application Publication
(10) Pub. No.: US 2023/0215063 A1
(43) Pub. Date: Jul. 6, 2023

(54) COMPUTER-IMPLEMENTED DETECTION AND PROCESSING OF ORAL FEATURES

(71) Applicant: ORAL TECH AI PTY LTD, Sandgate (AU)

(72) Inventors: Padma Gadiyar, Brisbane (AU); Praveen Narra, San Jose, CA (US); Anand Selvadurai, Tamil Nadu (IN); Guna Sekhar Thakkilla, Andhra Pradesh (IN); Muni Hemadri Babu Jogi, Andhra Pradesh (IN)

(21) Appl. No.: 18/000,987

(22) PCT Filed: Jun. 8, 2021
(86) PCT No.: PCT/IB2021/055048

§ 371 Date: Dec. 7, 2022

Related U.S. Application Data
(60) Provisional application No. 63/036,456, filed on Jun. 9, 2020.

(51) Int. Cl. G06T 11/60; G06V 40/16; G06T 7/73; G06V 10/82; G06T 7/90; G06T 7/12; G06T 7/00; G16H 30/40

(52) U.S. Cl. CPC G06T 11/60; G06T 7/12; G06T 7/0012; G06T 7/73; G06T 7/90; G06V 10/82; G06V 40/162; G06V 40/165; G06V 40/172; G16H 30/40; G06T 2200/24; G06T 2207/10024; G06T 2207/20021; G06T 2207/20084; G06T 2207/20104; G06T 2207/30036; G06T 2207/30201; G06T 2210/41

(57) ABSTRACT

Described herein are computer-implemented methods for identifying and classifying one or more regions of interest in a facial region and augmenting an appearance of the regions of interest in an image. For example, a region of interest may include one or more of: a teeth region, a lip region, a mouth region, or a gum region. User selected templates for teeth, gums, smile, etc. may be used to replace the analogous facial features in an input image provided by the user, for example from an image library or taken with an image sensor. The computer-implemented methods described herein may use one or more trained machine learning models and one or more algorithms to identify and classify regions of interest in an input image.

### CROSS-REFERENCE TO RELATED APPLICATIONS

This application claims the priority benefit of U.S. Provisional Pat. Application Ser. No. 63/036,456, filed Jun. 9, 2020, the contents of which are herein incorporated by reference in their entirety.

### TECHNICAL FIELD

This disclosure relates generally to the field of computer-implemented detection and processing applications, and more specifically to the field of automated image analyzing and processing.

### BACKGROUND

With the increased focus on social media photographs and images, and, in particular, photographs and images of our facial and bodily features, individuals recognize flaws in their personal appearance. There are software programs, such as Adobe Photoshop, that an individual may use to adjust or enhance an image, but these software programs require manual processing of images. More specifically, a person using a traditional software program needs to identify a first area requiring enhancing, and, using the software program, manually adjust a specific area. The user would then move to the next area in the image that needs adjusting or enhancing. This is a tedious process and, for untrained people of the software program, an even more difficult process.

Additionally, people request the services of cosmetic or reconstructive surgeons to alter their facial features. These surgeons may use example images to illustrate various ideas for individuals to choose from that represent their desired look or aesthetic, but those images are not of the individual themselves.

What is needed, therefore, is a computer-implemented software application that analyzes, processes, and alters images of individuals without requiring a manual manipulation of the image in order to achieve a desired look or aesthetic.

### BRIEF DESCRIPTION OF THE DRAWINGS

The aspects, features, and advantages of the present technology are described below in connection with various embodiments, with reference made to the accompanying drawings.

FIG. 1 is a flowchart of an overview of one embodiment of the present invention that is implemented on a mobile phone using a software application.

FIG. 2A illustrates an overview of the software application process, in accordance with an embodiment of the present invention.

FIG. 2B illustrates a block diagram of hardware components, in accordance with an embodiment of the present invention.

FIG. 3 is a flowchart of the software application used in a health environment, in accordance with an embodiment of the present invention.

FIG. 4 is an illustration of the application options of the software application used in a health environment, in accordance with an embodiment of the present invention.

FIG. 5 is a flowchart of a training workflow of a lip region identification model and individual teeth region identification model.

FIG. 6 illustrates a training architecture of the lip and individual teeth region identification model, in accordance with an embodiment of present invention.

FIG. 7 illustrates a table showing an example of the average precision (AP) of the prediction on the evaluation dataset at block S540 of FIG. 5 of a lip region identification model.

FIG. 8 illustrates a table of an example of the performance of a teeth identification model in accordance with an embodiment of the present invention.

FIG. 9 illustrates a graph showing the relationship of the precision and the recall of one embodiment of a Lip Region Identification model.

FIG. 10 is a flowchart of an example of an automated smile design workflow, in accordance with an embodiment of the present invention.

FIG. 11 illustrates the input image and the customized image, in accordance with the present invention.

FIGS. 12A-B is a flowchart illustrating the computer-implemented smile design workflow, in accordance with an embodiment of the present invention.

FIGS. 13A-Q illustrate various screenshots of examplary user interfaces, in accordance with the present invention.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

The computer-implemented system functions to assess and customize a facial feature of an image. The system is used to allow a user to design a customized smile according to their specific face, but can additionally, or alternatively, be used for any suitable dental or oral application. In some embodiments, the system functions to also provide users with personalized health and insurance information. The system can be configured and/or adapted to function for any other suitable purpose, such as enhancing or altering additional facial, oral, or dental features, and receiving varying health information associated with the additional facial, oral, or dental features. For example, the systems and methods described herein may function to identify and/or provide visual options for fixing one or more of: missing teeth, crooked teeth, broken or chipped teeth, discolored gums, receding gums, gaps between teeth, diseased teeth or gums, etc.

Any of the methods described herein may be performed locally on a user computing device (e.g., mobile device, laptop, desktop computer, workstation, wearable, etc.) or remotely (e.g., server, remote computing device, in the “cloud”, etc.).

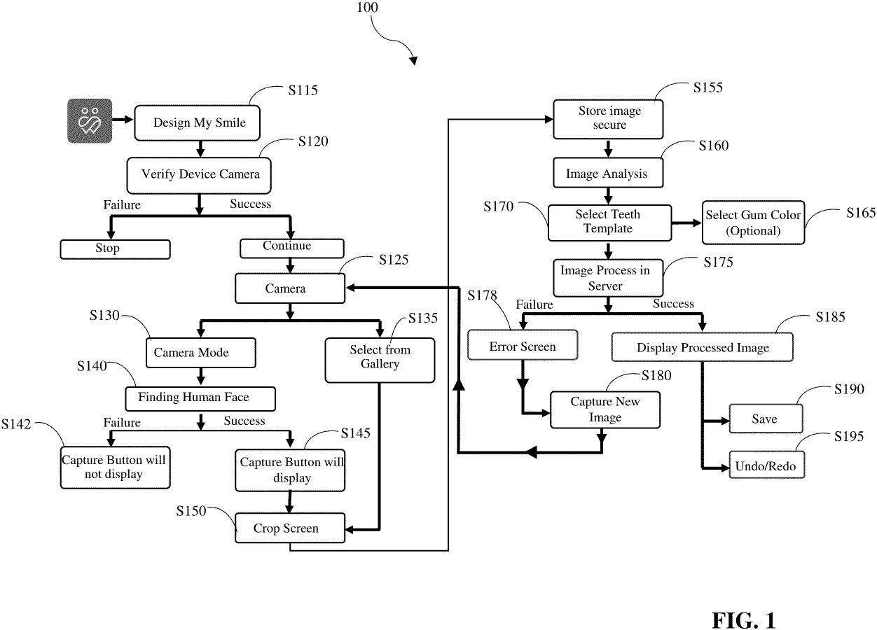

FIG. 1 shows a flowchart 100 of an overview of one embodiment of the present invention that is implemented on a mobile phone using a software application. It will be appreciated that a user can download the mobile application at any time. When a user is ready, the mobile software application is launched. In an exemplary embodiment of the present invention, the user is desiring to design their smile, at block S115, using the application. It will be appreciated, however, that other facial, oral, and/or dental features, may also be incorporated into the present invention. At block S120, the mobile application verifies a device camera at block S125, which prepares the application for camera mode at block S130. Alternatively, an image may be selected from a gallery or database of stored images at block S135, for example after the user accepts or acknowledges that the application can access the database. While in camera mode at block S130, the camera locates a face at block S140, discussed in detail further below, of either the user or another proximate individual. In one embodiment of the present invention, if there are alignment or focusing issues with locating a face, a capture button may not display, as shown at block S142; however, once the face is located, a capture button is displayed S145 on the graphical user interface (GUI). In some embodiments, the application may display an option for the user to crop the input image displayed on the screen at block S150 and save the input image at block S155 to local or remote memory.

Further, at block S160 of FIG. 1, a graphical user interface (GUI) of the software application notifies the user that the input image is being analyzed. Once the initial analysis is complete, the user is provided with a GUI that presents the user with selection options of various teeth templates at block S170 and/or optionally, one or more gum colors at block S165. The user selects and submits their desired options, and the software application processes these selections at block S175 in order to alter the original smile and/or gum color in the original image. In the event of errors in imaging at block S178, the software application is configured to allow the user to retake the image at block S180. The processed customized image is displayed at block S185, and the customized image is optionally saved, for example to a database in the application and/or to a database on the user’s mobile device at block S190. Alternatively, the application can return to the step of selecting the teeth and gum templates at block S195, for example in the event that the user did not like the previously selected templates.

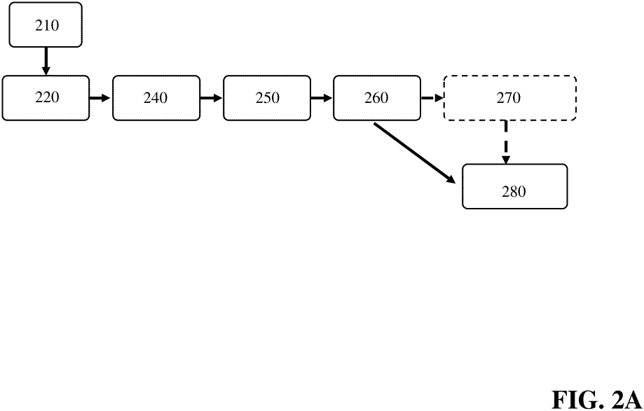

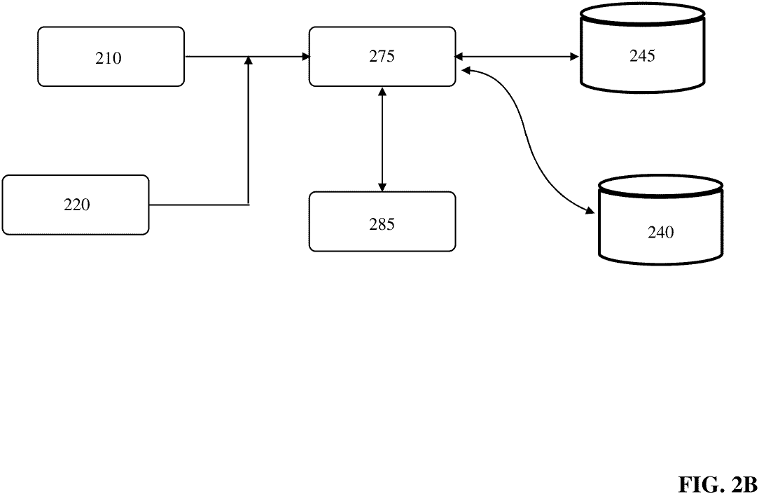

FIG. 2A, in conjunction with FIG. 2B, illustrates an overview of the software application process and a block diagram of hardware components in accordance with an embodiment of the present invention. A camera 210 is used to capture an image 220, for example using one or more audio visual frameworks or libraries of a computing device (e.g., AVFoundation or equivalents thereof), where the image may be of the user or another individual. It will be appreciated that the camera 210 may be integral to the overall system, such as a camera in a mobile phone, laptop, or computer, or the camera 210 may be a separate device that is capable of sending or uploading images to an external processor and memory. A cloud server 240 or other separate datastore 245 may be used for storing the image remotely and is later accessed by the mobile phone or computer. It will also be appreciated that the software application may reside and operate on either a local or remote processor 275 and memory 285, such as the mobile phone, laptop, ipad, or other computer processing unit. The local or remote processor 275 may either download the software application from a web address or disk, or the software application may run remotely. In one embodiment of the present invention, the input image 220 is received by processor 275, and processor 275 runs the software application stored in local memory 285. A preview of the input image as well as teeth style templates and/or gum shade templates 260 are displayed to the user for selection. In one configuration of the present invention, a machine-learning algorithm 270 is used to generate the templates. After the user selects their desired templates, the software application alters and customizes the input image according to selected templates and various parameters, and a resulting customized image 280 is displayed to the user. In some embodiments, the software application presents a user interface having a plurality of interaction screens associated with a processor. For example, an interaction screen may include an image input screen configured to receive an input image of at least one facial feature. Additionally, or alternatively, an interaction screen may include a selection interaction screen for presenting to the user a number of template variations corresponding to the at least one facial feature. Each template variation may include a plurality of template coordinates, such that the selection interaction screen is configured to receive a user selection of one or more of the template variations.

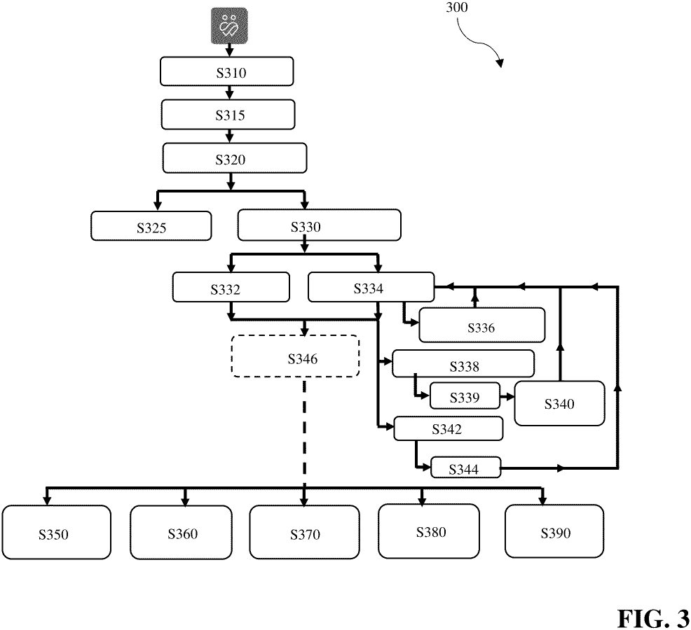

FIG. 3 shows a flowchart 300 of the software application used in a health environment, in accordance with an embodiment of the present invention. When the software application is initialized, an introductory splash screen is presented at block S310 and onboarding screen is displayed at block S315. At block S320, the application displays one or more user roles, linked to appropriate modules, for selection by a user. For example, a user may select a doctor login module at block S325 or a patient login module at block S330. If the user is a patient, the user may sign in at block S332 using a various social sign in, such as using a Google® or Facebook® account, or the user may sign in at block S334 using an email account. It will be appreciated that the user may also sign in using a sign in name or any equivalents thereof. At block S336, if the user does not have a current registered account or the account is not activated, the software application may return to blocks S332 or S334 to prompt a user to sign up for access to the software application at S342 by providing the user a one-time password (OTP) at block S344 and enter a password at block S334. Additionally, if the user has forgotten their password at block S338, the software application may provide a one-time password (OTP) at block S339, prompt the user to create a new password at block S340, and resend the user to blocks S334 to enter the new password. In some embodiments, the application may optionally prompt the user to allow push notifications at block S346.

Once the user has signed into the software application, the graphical user interface displays one or more application options for selection. Some example options may include, but are not limited to: an oral health score at block S350, the design my smile at block S360 (shown in FIG. 1), awareness at block S370, reminders at block S380, or a menu option at block S390.

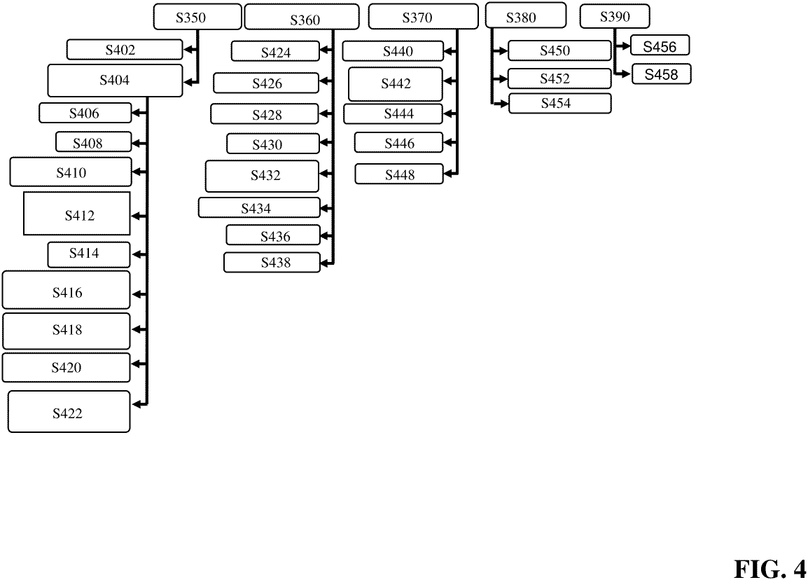

FIG. 4 shows an illustration of the application options of the software application used in a health environment, in accordance with an embodiment of the present invention. If the user selects an oral score at block S350, the software application interacts with the user to formulate an overall oral health report (e.g., based on one or more of: health, dental history, current dental image analysis, etc.). Advantageously, the software application helps guide the user towards appropriate hygiene choices. More specifically, the software application initially interacts with the user to provide introductory videos at block S402, instructional videos at block S408, help videos at block S406; instructions on how to use the oral health score report at block S404; and receive teeth images of the user, e.g., from a camera or a stored image at block S410. The image may be optionally cropped or processed, for example using one or more filters or tools in an editing application. After analyzing the uploaded photograph at block S412, the software application provides an oral report at block S414. More specifically, the software application uses artificial intelligence and training libraries to analyze the image of the user’s teeth and/or gums and calculate the presence or absence of dental caries, periodontitis, an impacted tooth or teeth, hyperdontia, gingivitis, oral cancer, abscessed tooth or teeth, bleeding gums, or other oral health conditions or oral diseases. These calculations provide the user with an oral health rating that the user can then use when visiting a dentist or periodontist. Further, the software application may optionally present questions related to the user’s hygiene. For example, the user is asked one or more oral hygiene questions at blocks S416 or follow-up questions (e.g., based on answers to questions at block S416) at block S418. In one embodiment based on their answers, the software application will then display how the answers are ranked (e.g., ranking guidelines) at block S420 and provide an overall oral health score report at block S422.

The user may also select the design my smile option at block S360. The software application provides an introduction at block S424 to this portion of the software and initializes the camera mode at block S426. Alternatively, the user may select to load an input image from an image library or gallery at block S428. The application may optionally crop the input image to reflect a subset region of the input image for designing at block S430. For example, the user may want to design their smile and teeth, and the input image is cropped to display that region. It will be appreciated, however, that while the drawing reflects a smile, the software application can accommodate any other dental or oral feature, such as the user’s lips, gums, teeth, tongue, etc. The software application analyzes the input image at block S432 and interacts with the user to alter, adjust or enhance their smile at block S434, and the altered customized image is saved at block S438. If there are any input image errors at block S436, the user is notified.

The user may select the awareness option at block S370 when the user is interested in educational information. The educational materials may include, but not be limited to, recent articles (e.g., on health topics, sleep habits, dental care habits, etc.) at block S440, rankings of most-like articles at block S442, article details at block S444, etc. The user may be able to share those articles by liking them or sharing them with others at blocks S446, S448.

Further, the user may select the reminders option at block S380. The user may advantageously have a reminders list at block S450 by adding at block S452 and/or editing reminders at block S454. These reminders can be related to any health reminder, such as timers for brushing their teeth, visiting a dentist, reminders to floss, reminders to not chew nails or ice, for example.

Additionally, the user may select the menu option at block S390 where the user may complete their profile at block S456 including any personal, medical or lifestyle information. The user may set their password and other account information. There are various forums in which the user may participate at block S458. The user may be able to view all posts, his/her posts, search posts, add new posts, post details, add comments, like posts, share posts, etc. Further, there is other information stored that is related to the software application and its use.

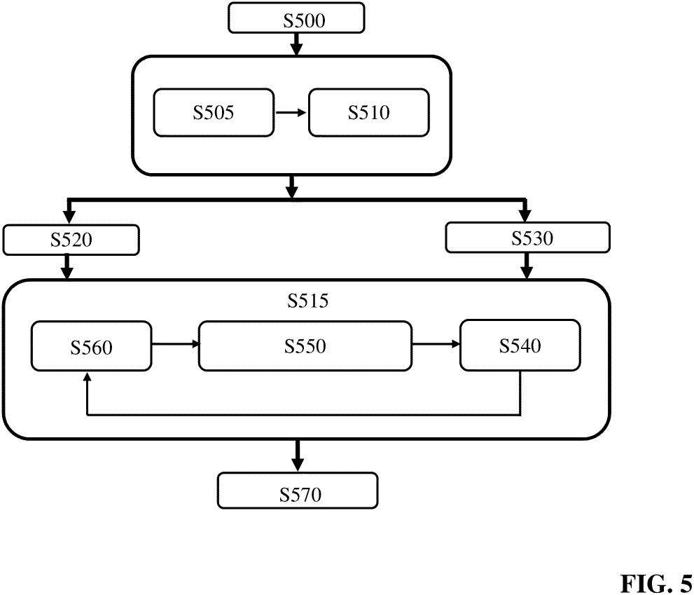

FIG. 5 shows a flowchart of a training workflow of a lip region identification model and individual teeth region identification model. A machine-learning system may be trained using an original dataset at block S500 to identify lip regions and individual teeth regions. A plurality of images is segmented into regions of interest (ROI) at block S505 and annotated at block S510. The annotated ROIs of the images are divided into training datasets at block S520 and evaluation datasets at block S530. The software application is then trained at block S515 using these datasets to train a machine learning model at block S550 by evaluating the dataset at block S540 and updating the weights of the models at block S560. In one embodiment, a Mask R-CNN architecture is used at block S550. Other alternatives for the Mask R-CNN include, but are not limited to: U-Net and Fully Convolutional Network (FCN). Architectures like Mask R-CNN may work as a combination of two networks in which one network is used to detect an object in the image like object detection and another network outputs an object mask which is a binary mask that indicates the pixels where the object is in the bounding box. Each pixel in the ROI is then classified and annotated in order to output masks for the identified objects in the image. In alternate embodiments, other models like Object Detection may be used, such that the ROI is classified as a whole single object. After the training, the best template model is saved for future processing at block S570.

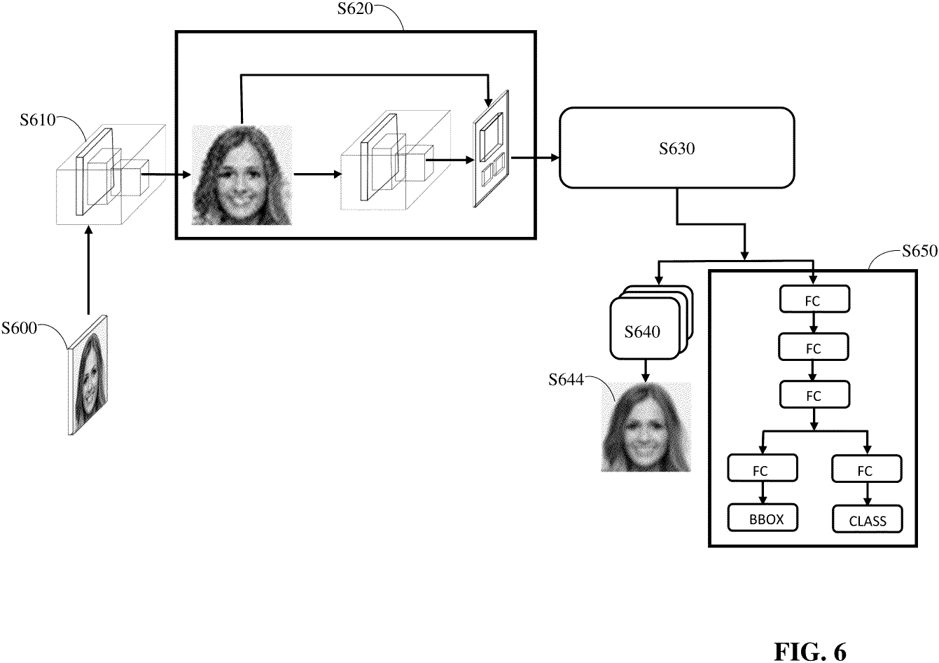

FIG. 6 illustrates a training architecture of the lip and individual teeth region identification model in accordance with an embodiment of present invention. For training one or more segmentation models herein, various architectures may be used which in turn use various types of backbone networks to perform one or more tasks. Methods disclosed herein may comprise the step of detecting bounding boxes of an object. This may be performed using ResNet (Residual Network), EfficientNet, Inception networks, etc. Methods disclosed herein may comprise the step of detecting the object’s mask from the bounding box. This may be performed using different networks including, but not limited to FPN (Feature Pyramid Network), DC5 (Dilated-C5) networks, etc. One or more models may be trained with combination of networks. As shown in FIG. 6, an input image is received by the application at block S600. The image is segmented (e.g., using object detection, localization, and classification) to identify one or more objects therein (e.g., facial features, lips, teeth, nose, etc.). At block S610, bounding boxes of each of the one or more objects are detected, for example using ResNet and FPN. At block S620, a Region Proposal Network (RPN) generates estimates or ‘proposals’ for regions in which objects may be positioned and uses a classifier to determine the probability of whether a proposal or estimate includes an object (e.g., lips, teeth, etc.). The RPN uses a sliding window method to determine relevant anchor boxes (i.e., pre-calculated bounding boxes of different sizes that are placed throughout the image that represent the approximate box predictions so as to save the time to search) from the feature maps. The anchors are classified in a binary fashion for whether the anchor has the object or not and then bounding box regression is performed to refine bounding boxes. The anchor is classified as positive label if the anchor(s) has highest Intersection-over-Union (IoU) with the ground truth box, or it has IoU overlap greater than 0.7 with the ground truth box.

The top positive anchors, Regions of Interest (ROIs), are output by the RPN. At block S630, ROIs are aligned. For example, features are transformed from the ROIs (which have different aspect sizes) into fixed size feature vectors without using quantization. The ROIs are aligned by bilinear interpolation, in which a grid of sampling points is used within each bin of ROI to interpolate the features at its nearest neighbors. For example, a max value from the sampling points is selected to achieve the required feature map. Further, at block S640, a convolutional layer receives the feature map and predicts masks (e.g., pixel-to-pixel alignment) at block S644. At block S650, one or more fully connected (FC) layers receive the feature map and predict class score (e.g., lip, teeth, gums, etc.) and bounding box (bbox) offset for each object.

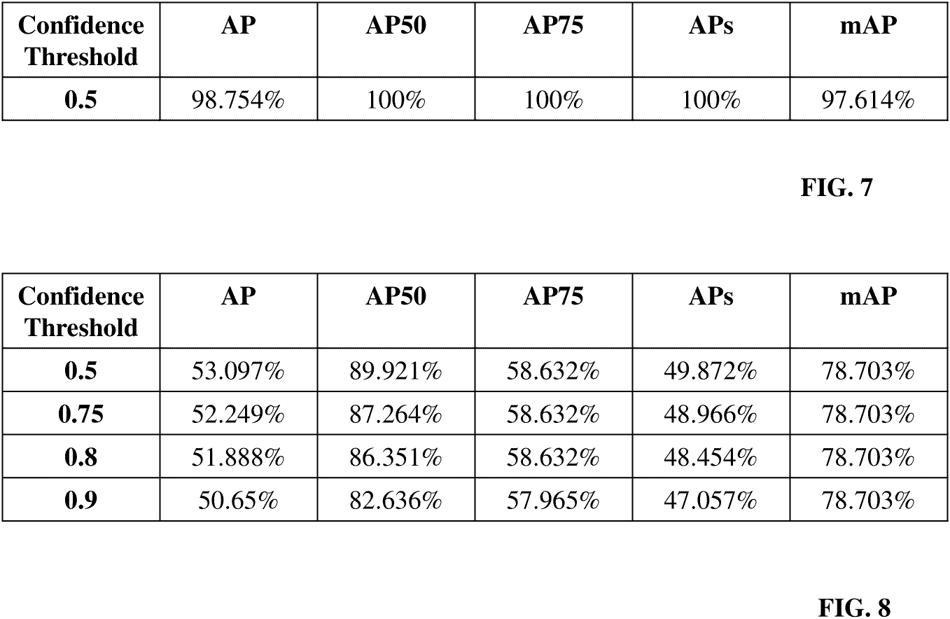

FIG. 7 shows a table showing an example of the average precision (AP) of the prediction on the evaluation dataset at block S540 of FIG. 5 of a lip region identification model. In one embodiment, the output from the model is filtered using a confidence threshold to reduce false predictions and only predictions with more than a predetermined confidence level (e.g., 90% confidence) are considered. The prefix after AP (e.g., AP50) represents the IoU threshold considered for calculating the Average Precision. For example, AP50 is an IoU threshold of 0.5, and AP75 is an IoU threshold of 0.75. Further, s, m, and 1 represent a scale of the Average Precision. For example, APs is percent Average Precision small scale for small objects having a predicted area less than about 32 squared. Further for example, APm is percent Average Precision medium scale for medium objects having a predicted area between about 32 squared and about 96 squared. Still further, for example, APl is percent Average Precision large scale for large objects having a predicted area greater than about 96 squared. Area is measured as the number of pixels in the segmentation mask. FIG. 8 illustrates a table of an example of the performance of a teeth identification model in accordance with an embodiment of the present invention and the description above for FIG. 7. In the embodiment of FIG. 8, multiple pre-determined confidence levels were considered.

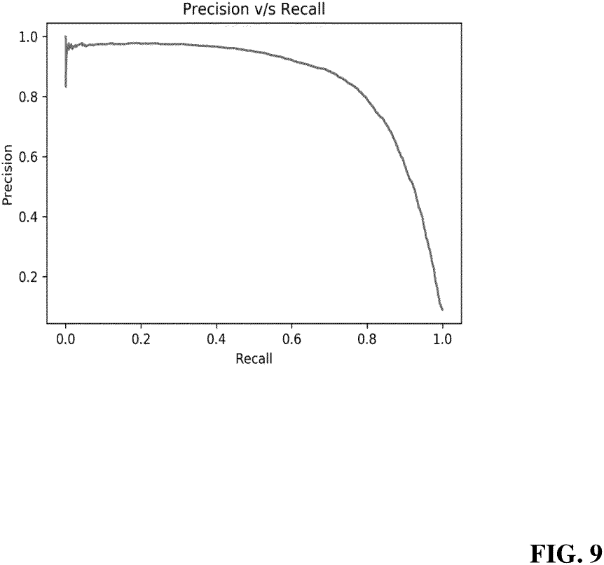

FIG. 9 shows a graph showing the relationship of the precision and the recall of one embodiment of a Lip Region Identification model. The average precision (AP) is equal to the area under precision-recall curve, where: precision = TP/(TP+FP); recall = TP/(TP+FN); TP = True positives; TN = True Negatives; FP = False Positives; FN = False Negatives.

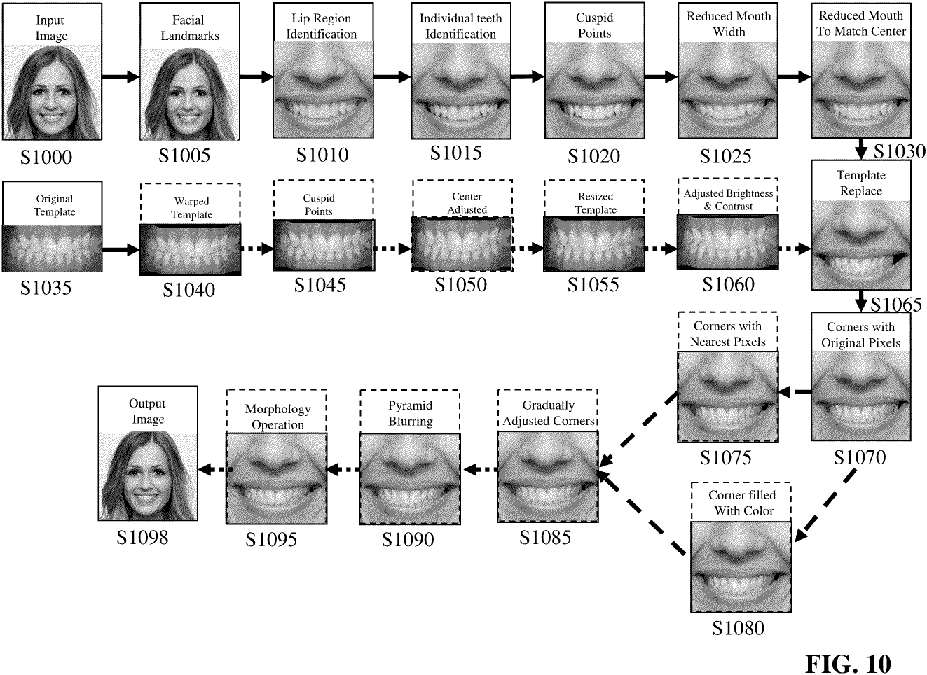

FIG. 10 is a flowchart of an example of an automated smile design workflow, in accordance with an embodiment of the present invention. It will be appreciated that the software application performs a series of steps to analyze and determine parameters of the input image and provide a customized image of a designed smile. Parameters of the input image may include one or more of: facial landmarks, lip regions, teeth identification, cuspid points, mouth corners, brightness, contrast of the input image, equivalents thereof, or combinations thereof. Initially, an image is uploaded at block S1000 to the software application. Alternatively, an image sensor, such as a camera, is used to capture the input image of a facial feature. The software application detects the presence of a face in the image and analyzes the image to extract one or more facial landmark points at block S1005.

In one embodiment, when using an image sensor to capture the face, a dlib frontal face detection model may be used to detect and localize the face in the input image. The graphic user interface may be configured to allow the image sensor to capture the image of the face once the face is detected. In other embodiments of the present invention, custom-built, dedicated models or other applications, such as facial libraries or Apple Vision Framework®, may be used to detect and localize the face. It will be appreciated that any application can be used to identify the features of a face, such as eyes, nose, and teeth.

Further, a dlib shape predictor algorithm may be used to locate and map key facial landmark points along a shape of one or more regions of interest, such as eyes, eyebrows, nose, mouth, lips and jawline. The dlib shape predictor utilizes trained models to estimate the location of a number of coordinates (x, y) that map the user’s facial landmark points in the image. The facial landmark points are then used to determine the user’s positional view of the face (e.g., a frontal head pose), alignment of the face, corners of the mouth, lip regions, or combinations thereof. In one embodiment, the face alignment, and its corresponding coordinate points, is a suitable reference to use in order to align and swap the teeth in the input image to the selected teeth template. Further, a rotation of the image, based on, for example, eye coordinates extracted from the facial landmark points (e.g., using dlib shape predictor algorithm), may also be performed. More specifically, in an embodiment of the present invention, the dlib shape predictor algorithm calculates the coordinate points for both the right and left inner corners of the eyes, and the input image is then rotated in such a way that a vertical difference between a center point of each of the eye coordinates is minimized or reduced to zero. Advantageously, rotation of the image, if necessary, helps to prevent or reduce misalignment of the teeth template with respect to the teeth of the input image. After an input image is rotated, the facial landmark points will change and may need to be updated. To obtain the changed facial landmark points for the rotated image, the software application will again detect the ROI (Region of Interest) for the face and, using the dlib shape predictor algorithm, calculate the facial landmark points for the rotated image. It will be appreciated that other algorithms or software applications may be used to calculate the facial landmark points in the input image, including: a deep learning model or Apple Vision Framework®.

At block S1010, the software application identifies the lip region. Using the identified facial landmark points and the trained lip region identification algorithm, as described above in connection with FIGS. 5-6, the software application identifies the lip regions (inner lip region, outer lip region, surface lip region, etc.) and corners of the mouth at block S1010. At block S1015, the software application identifies each individual visible tooth using the trained teeth region identification algorithm, as described above in connection with FIGS. 5-6. At block S1020, the software application also identifies the canine teeth and their cuspid coordinate points in the input image. In one embodiment of the present invention, the identified cuspid coordinate points of the input image may be used as reference coordinate points that are mapped to the cuspid coordinate points of the teeth template. More specifically, the segmented input image identifies the cuspid coordinate points, and the cuspid coordinate points of the teeth template are known. In this manner, the software application uses these coordinates to replace the teeth of the input image with the selected teeth style of the teeth template. Other, non-limiting parameters that are calculated from the cuspid coordinate points of the input image are symmetry of the teeth, facial and dental midlines, tooth dimensions, etc. For example, generally cuspid points are equally located at on both sides of the facial and dental midline. As such, the cuspid coordinate points may be used to calculate symmetry. It will be appreciated that, if there are broken, worn down, or missing cuspid points, other teeth may be used to map the template to the input image. At block S1025, the software application may reduce the mouth width, and, at block S1030, the software application matches the reduced mouth width to match at the center of the mouth.

Further, the software application begins processing the selected teeth style selected by the user at block S1035. In particular, the software application may optionally adjust one or more parameters and coordinates of the selected teeth style template to match the parameters and coordinates of the input image. These optional adjustments may include one or more of: warping, or bending (or altering a shape of the template), a teeth template at block S1040 in order to better fit the teeth style template over the mouth of the input image; identifying the cuspid coordinate points of the template at block S1045 in order to match and center the midpoint of the cuspid coordinate points of the template to the midpoint of the cuspid coordinate points in the input image at block S1050; and/or resizing the teeth template to match size of the input image at block S1055. If the teeth template requires resizing to fit appropriately into the mouth of the input image, ratio values are calculated using the lip region identification model and the facial landmark points, as described above. The software application may also optionally adjust the brightness and contrast of one or more portions of the template at block S1060. The adjusted teeth template is then applied to the input image at block S1065 using the parameters of the input image, thereby replacing the teeth of the input image with the teeth template, to produce an altered input image.

At block 1070, the software application analyzes the corners of the mouth and lip regions in the altered input image for any empty corridor regions, or enlarged dark areas between the teeth and the lips, in the mouth. Any empty corridor regions of the mouth can optionally be filled with nearest pixel values at block S1075 and/or filled with an average color value of the corresponding area of the input image at block S1080. It will be appreciated that one, or both, or none of these alterations are necessary for processing the altered input image. All corners of the mouth can also be gradually adjusted at block S1085 in such a way that from the outer to the inner portion of the mouth corridor gradually reduces the importance for the original input image and increases the importance for the teeth template. Pyramid blurring at block S1090 optionally smooths the transition from the input image to the altered input image, and a morphological operation at block S1095 may also optionally be applied on the mouth region to remove any noise generated while performing the computer-implemented process. The morphological operation may include one or more of: mathematical morphology, convolution filtering, noise reduction, or a combination thereof. For example, mathematical morphology is an image processing technique based on two operations: erosion and dilation. Erosion enlarges objects in an image, while dilation shrinks objects in an image. Convolution filtering involves taking an image as input and generating an output image where each new pixel value is determined by the weighted values of itself and its neighboring pixels. Noise reduction takes an image as input and removes all unnecessary elements in that image so that it looks better. The altered input image may require the final steps of reverting back a rotated input image, if rotation was performed, into its original shape, angle, and/or resolution and saving the final customized image at block S1098.

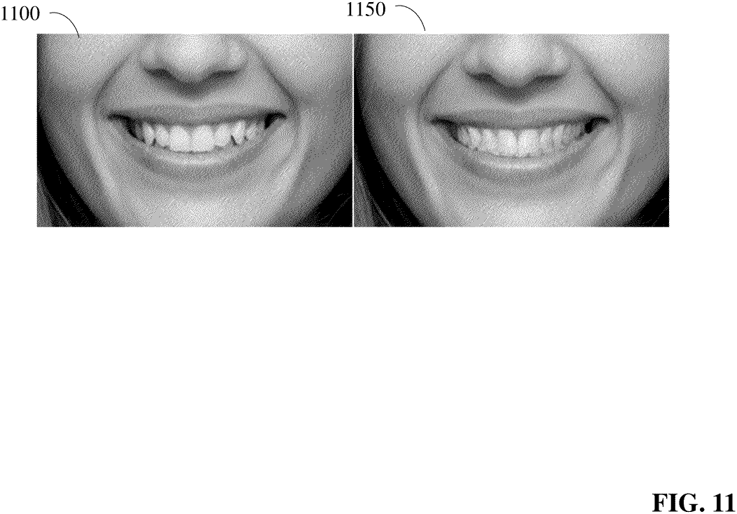

FIG. 11 illustrates the input image 1100 and the customized image 1150, in accordance with the present invention. As illustrated in this example, the user chose a particular teeth style at block S1035 of FIG. 10. The user may have also chosen a gum color template. If the user did not choose a particular gum color, it will be appreciated that the software application may leave the color as the original color, or it may shade the gum color an appropriate color that matches the lip regions. As illustrated in the customized image, the user may use this smile design for their personal reasons, or they may show this smile design and teeth style to a cosmetic or reconstruction surgeon as an illustration of their desires in a physical transformation.

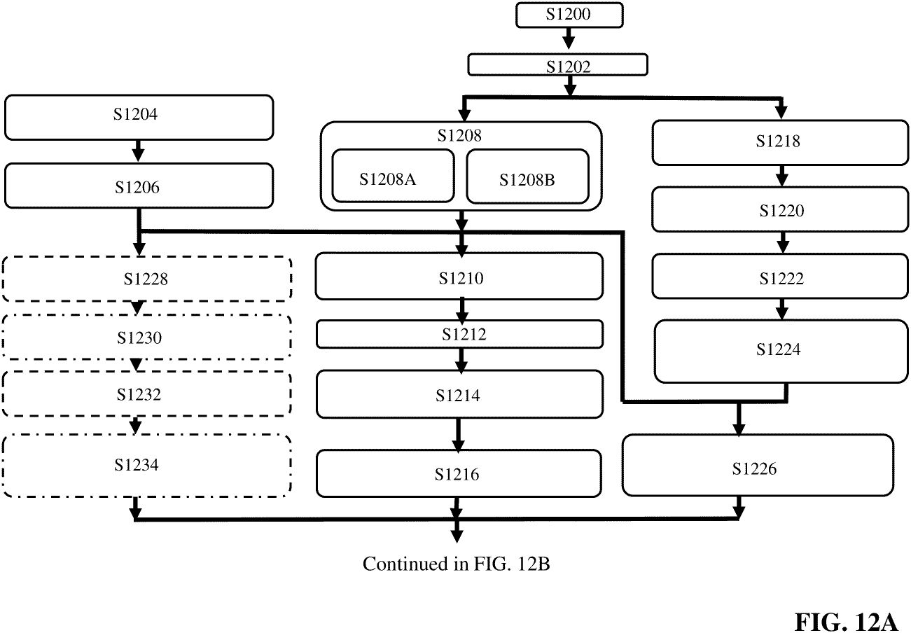

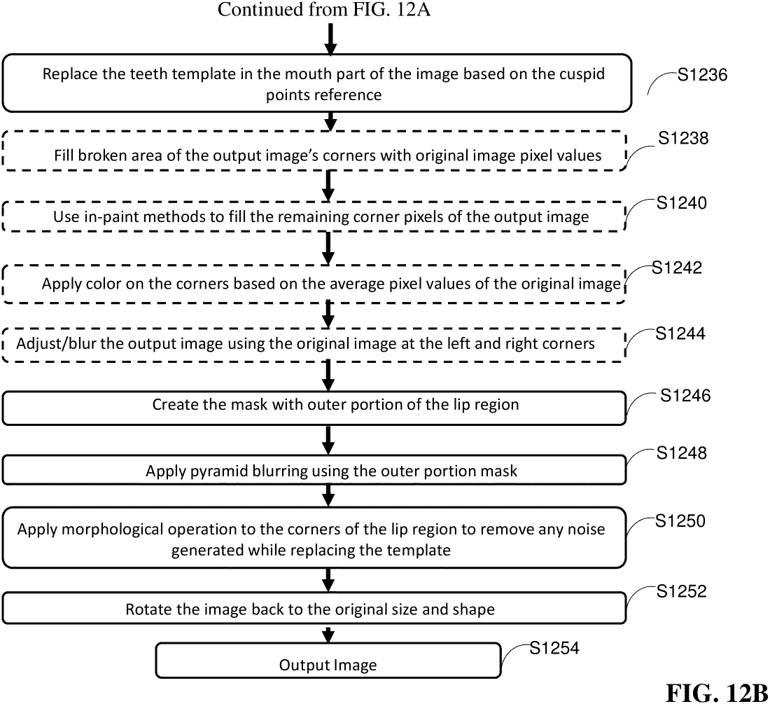

FIGS. 12A-B is a flowchart illustrating the computer-implemented smile design workflow in accordance with an embodiment of the present invention. In conjunction with FIG. 10, FIGS. 12A-B illustrates the steps for receiving an input image and providing a customized image. The input image of a face with a smile showing teeth is received at block S1200, and the image data is read at block S1202. Simultaneously or separately, the software application performs a plurality of steps. A collection of teeth templates and gum shades are provided to the user for selection at block S1204. The selected templates are received S1206, and the template data is read. The teeth style and gum shade will be used to apply to the input image.

A machine-learning algorithm analyzes the input image at block S1208 and predicts the lip region at block S1208A and/or the individual teeth region at block S1208B for the subset region of the facial feature. Using a lip region identification model, a mask is created for the lip region of the input image at block S1210. The input image is segmented and, focusing on the ROI of the lip region, the top pixels are determined at block S1212. At block S1214, the top pixels are adjusted in the lip region in order to reduce the pixelate area to replace the teeth of the input image with the teeth template. In some embodiments, the top cuspid teeth center points are determined using the lip region and individual teeth identification models at block S1216.

The software application detects the facial landmark points at block S1218 in the input image. If the facial landmark points are tilted, the input image is rotated at block S1220 to adjust for the tilt. In this case, the facial landmarks points are detected again at block S1222. The left and right corners of the mouth are determined at block S1224. Using the corners of the mouth, the area of the lip region is reduced in size at block S1226, optionally or if necessary.

Further, after the user has selected the teeth style template and optional gum shade, the software application alters the teeth style template to match the determined parameters of the input image, such as size of the teeth, lip regions, cuspid teeth, and midpoints of the mouth. It will be appreciated that the teeth style template may be require very little to no alterations or drastic alterations in order to match the input image. Some of the alterations may not be required at all. If necessary, however, based on the parameters of the input image, alterations to the teeth style template may include one or all of the following: warping the teeth template at block S1228, adjusting the template midpoint to match the midpoint of the input image at block S1230, resizing the teeth template to fit into the width of the mouth of the input image at block S1232, and adjusting the brightness and contrast to match the brightness and contrast of the input image at block S1234.

Referring now to FIG. 12B, the cuspid points of the input image are used as a reference to replace the teeth of the input image with the altered teeth template at block S1236. Optionally, any broken areas in the corners of the mouth are filled with pixel values at block 1238. Optionally, the user can manually fill in the corner pixels using a painting method at block S1240. Alternatively, and optionally, the software application can apply color values to the corners based on an average pixel value of the original input image at block S1242. All corners of the mouth can also be gradually adjusted at block S1244 in such a way that from the outer to the inner portion of the mouth corridor gradually reduces the importance for the original input image and increases the importance for the teeth template. A mask is created with the outer portion of the lip region at block S1246. Pyramid blurring at block S1248 smooths the transition using the outer portion mask, and a morphological operation at block S1250 may also be applied on the mouth region to remove any noise generated while performing the computer-implemented process. The altered input image may require the final steps of reverting back the rotated input image at block S1252, if rotation was performed, into its original shape, angle, or resolution and saving the final customized image at block S1254.

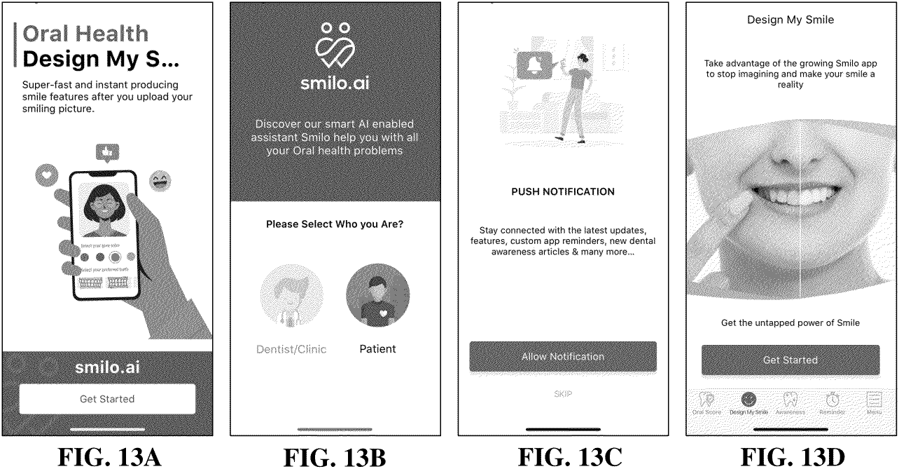

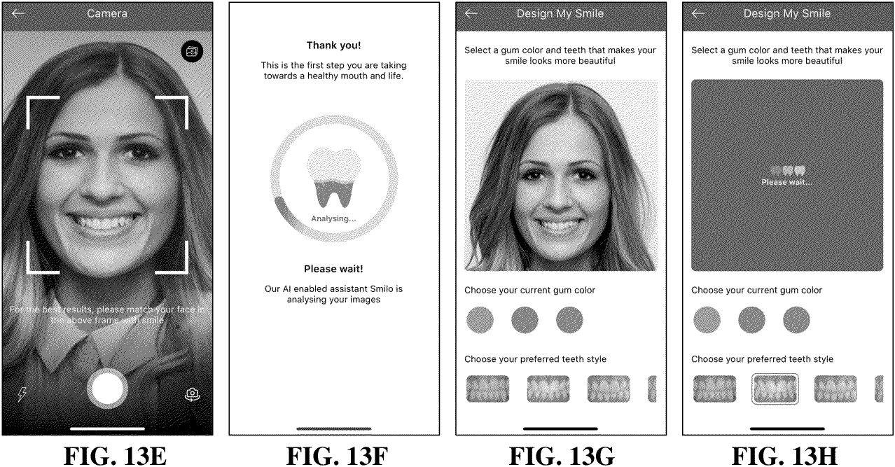

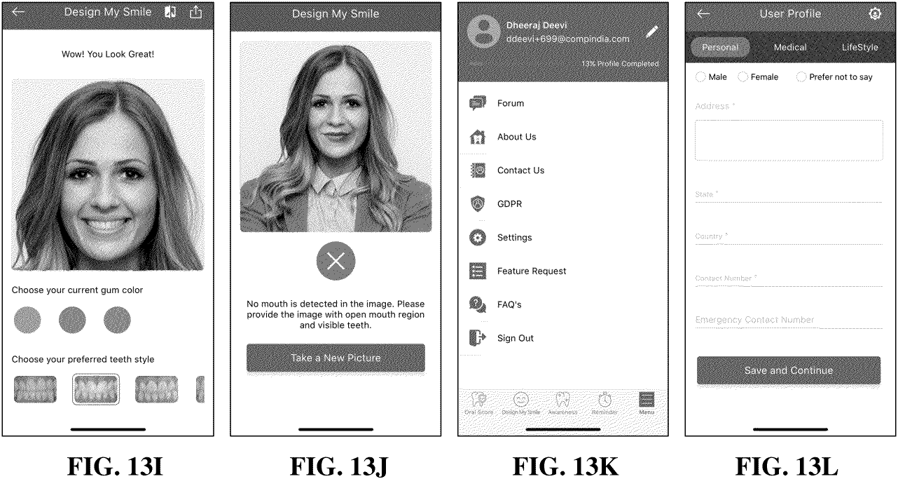

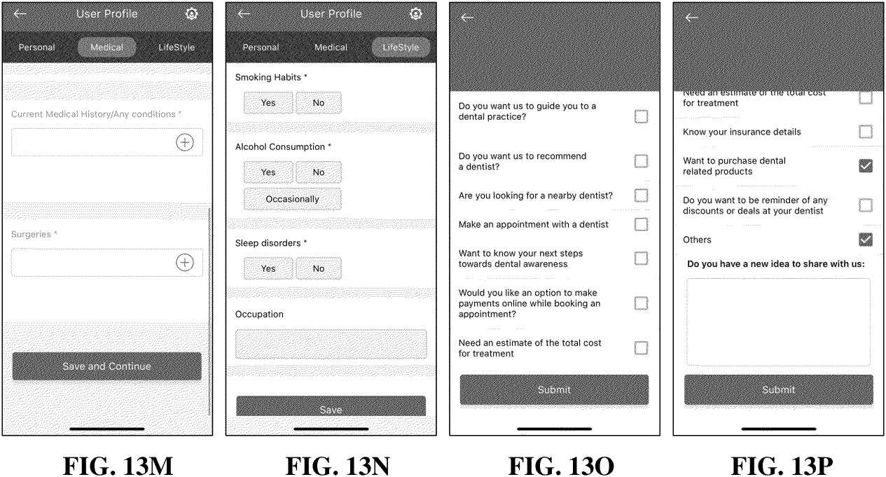

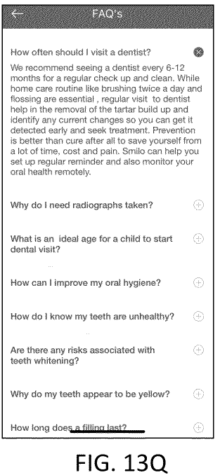

FIGS. 13A-Q illustrate various screenshots of an example user interface in accordance with the present invention. FIG. 13A is an example screenshot of on oral health and design my smile software application where a user launches the software application and selects the get started button. FIG. 13B is an example screenshot allowing the user to select their role (as in block S320 of FIG. 3) while using the software application. For example, a doctor, dentist, clinician, or patient. FIG. 13C is an example screenshot asking the user’s preference regarding notifications and updates (e.g., as in block S346 of FIG. 3). FIG. 13D is an example of the design my smile screenshot that allows the user to input an image, and the software application designs their smile (e.g., as described in FIGS. 10-12B). As shown in FIG. 13E, a camera of the mobile device is activated and a user positions his/her, or another’s, face within the borders (e.g., as described in FIGS. 1-2). An image is taken or selected from a database or library, and FIG. 13F shows an example screenshot of a notification presented to the user that the software application is analyzing the received input image. As shown in FIG. 13G, one or more teeth style templates and/or gum shades are displayed to the user for selection. After selection, FIG. 13H begins processing the selections based on the parameters of the input image and the selected teeth style and/or gum shades. FIG. 13I shows a graphical user interface displaying the customized image. In the event, the mouth is not showing or is out of focus, FIG. 13J is an example notification presented to the user indicating one or more errors.

FIGS. 13K-13N are example screenshots of a user’s profile that can be edited and updated for reference (e.g., as shown and/or described in connection with FIG. 4). For example, the user’s personal information (FIG. 13L), medical information (FIG. 13M), and lifestyle information (FIG. 13N) is entered into the software application. FIGS. 13O-13P are example screenshots asking the user questions related to oral health, such as recommendations of dental practices, making appointments, paying bills, or cost estimates, as well as insurance information. FIG. 13Q is an example screenshot of frequently asked questions that the user may read for reference.

It will be appreciated that the present invention can be used for various reasons, such as customizing their smile, receiving oral health information, or visualizing changes using their face for cosmetic or reconstructive purposes. Advantageously, the software application provides the customized image automatically without the need for the user to manually edit the images.

The systems and methods of the preferred embodiment and variations thereof can be embodied and/or implemented at least in part as a machine configured to receive a computer-readable medium storing computer-readable instructions. The instructions are preferably executed by computer-executable components preferably integrated with the system and one or more portions of the processor on the computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

Various embodiments will now be described.

One aspect of the present disclosure is directed to a computer-implemented method for assessing or at least partially reconstructing an image of one or more facial regions of a user. The method may include receiving, at a processor, an input image of at least a portion of a face; using one or more trained machine learning algorithms configured to: segment the input image into one or more regions, identify which of the one or more regions are a region of interest, and classify the regions of interest into one of: a mouth region, a lip region, a teeth region, or a gum region; using a shape predictor algorithm configured to identify a location of the one or more classified regions of interest in the input image; receiving, at a display communicatively coupled to the processor, a user input selection of a template comprising a desired aesthetic for one or more of the classified regions of interest; applying one or more characteristics of the selected template to the input image; and outputting an output image comprising the desired aesthetic of the one or more regions based on the selected template and said applying.

In any one of the preceding embodiments, the method may further comprise aligning a midpoint of the selected template with a midpoint of the region of interest.

In any one of the preceding embodiments, the portion of the face comprises one or more of: a mouth, one or more teeth, a nose, one or both lips, or a combination thereof.

In any one of the preceding embodiments, outputting the output image having the desired aesthetic further comprises outputting the output image having a desired smile appearance.

In any one of the preceding embodiments, the method further comprises providing one or more educational materials related to health of the facial region.

In any one of the preceding embodiments, the method further comprises: receiving one or more user inputs related to hygiene of the one or more facial regions; ranking the one or more user input based on a health guideline; and generating an oral health score report based on said ranking.

In any one of the preceding embodiments, the one or more trained machine learning algorithms comprise a mask R-Convolutional Neural Network architecture using a Residual Network and Feature Pyramid Network backbone.

In any one of the preceding embodiments, the shape predictor algorithm is a dlib shape predictor algorithm.

In any one of the preceding embodiments, the one or more identified regions comprise: a lip region, individual teeth, a cuspid point, a right corner position of a mouth, a left corner position of the mouth, a mouth coordinate position, a mouth area, or a combination thereof.

In any one of the preceding embodiments, applying comprises using the cuspid point as an initial reference to apply the template to a mouth region in the image.

In any one of the preceding embodiments, applying further comprises: warping the template, resizing the template for a best fit to the body region in the input image, adjusting one or both of a brightness or a contrast of the template to match with the input image, replacing the template in the body region, or a combination thereof.

In any one of the preceding embodiments, the classified region is a gum region such that the method further comprises identifying a gum color of the gum region in the input image and applying a desired gum color to the input image.

In any one of the preceding embodiments, the classified region is the mouth region, such that the method further comprises filling one or more corridors of the mouth region with nearest pixel values.

In any one of the preceding embodiments, the method further comprises displaying one or more guides for positioning of the at least a portion of the face in the input image.

In any one of the preceding embodiments, the method further comprises outputting an error message when the one or more features are out of a predetermined range.

Another aspect of the present disclosure is directed to a computer-implemented application system for assessing and customizing a facial feature of an image. The application may comprise: a user interface having a plurality of interaction screens associated with a processor, the user interface configured to receive user interaction; an image input screen configured to receive an input image of at least one facial feature; a selection interaction screen for presenting to the user a number of template variations corresponding to the at least one facial feature, each template variation having a plurality of template coordinates; and an output image interaction screen configured to present the customized image to the user.

In any of the preceding embodiments, the selection interaction screen is configured to receive a user selection of one or more of the template variations.

In any one of the preceding embodiments, the processor may be configured to alter the at least one facial feature of the input image based on the one or more selected template variations, and provide a customized image.

In any one of the preceding embodiments, the processor is configured to identify a plurality of input image coordinates to use as reference points for mapping to the plurality of template coordinates of the selected one or more template variations.

In any one of the preceding embodiments, the processor is configured to identify the plurality of input image coordinates by segmenting the input image into at least one region of interest, identifying boundaries of objects in the input image, and annotating each pixel based on the identified boundary.

In any one of the preceding embodiments, the plurality of input image coordinates is facial landmark points corresponding to one or more of: a lip region, individual teeth, cuspid points, a right corner position of a mouth, a left corner position of the mouth, a mouth coordinate position, a mouth area, a left eye, a right eye, or a combination thereof.

In any one of the preceding embodiments, wherein the input image is provided using one or both of: an input image sensor for taking and uploading the input image of the facial feature of the user or uploaded from an image library.

In any one of the preceding embodiments, the at least one facial feature of the input image comprises one or more of: a mouth, one or more teeth, gums, one or both lips, or a combination thereof.

In any one of the preceding embodiments, the number of template variations comprises one or more of: a number of varying gum shades or a number of varying teeth style templates.

In any one of the preceding embodiments, the selection interaction screen is configured to receive a user selection of one of the varying teeth style templates.

In any one of the preceding embodiments, the processor is configured to alter the selected teeth style template based on the plurality of coordinates of the selected teeth style template and the corresponding identified plurality of input image coordinates including one or more of: warping the selected teeth style template for a best fit to the facial feature of the input image, resizing the selected teeth style template for a best fit to the facial feature of the input image, adjusting one or both of a brightness or a contrast of the selected teeth style template to match a brightness or a contrast of the input image, or a combination thereof.

In any one of the preceding embodiments, the altered selected teeth style template replaces the facial region of the input image.

In any one of the preceding embodiments, the processor is further configured to analyze the teeth and gums of the input image, and calculate an oral health score based on one or more of: a presence or absence of dental caries, or gum disease; and provide the oral health score to the user.

In any one of the preceding embodiments, the processor is further configured to display on a display one or more educational materials related to a health of the facial region.

Another aspect of the present disclosure is directed to a method for customizing a facial feature of an image. The method may further comprise: receiving, at a processor, an input image having a facial feature identified for customization; identifying, at the processor, a plurality of facial landmark coordinates for the input image; presenting to a user a plurality of teeth style templates; receiving, at the processor, a selection of one of the teeth style templates; altering the plurality of coordinates of the selected teeth style template to match the plurality of facial landmark coordinates of the input image; and replacing the teeth region of the input image with the altered teeth style template to provide a customized output image.

In any of the preceding embodiments, the facial feature is one or both of: a lip region and a teeth region.

In any of the preceding embodiments, the plurality of facial landmark coordinates corresponds to one or more of: a lip region, a teeth region, cuspid points, a right corner position of a mouth, a left corner position of the mouth, a mouth coordinate position, a mouth area, a left eye, a right eye, or a combination thereof.

In any of the preceding embodiments, the selected teeth style templates comprise a plurality of coordinates.

In any of the preceding embodiments, replacing the teeth region comprises mapping cuspid point coordinates of the selected teeth style template with the cuspid point coordinates of the input image.

In any of the preceding embodiments, altering the selected teeth style template includes one or more of: warping the selected teeth style template for a best fit to the facial feature of the input image, resizing the selected teeth style template for a best fit to the facial feature of the input image, adjusting one or both of a brightness or a contrast of the selected teeth style template to match a brightness or a contrast of the input image, or a combination thereof.

In any of the preceding embodiments, the method further comprises: analyzing, at the processor, the teeth and gums of the input image, and calculating an oral health score based on one or more of: a presence or absence of dental caries, or gum disease; and providing the oral health score to the user.

In any of the preceding embodiments, the method further comprises providing one or more educational materials related to a health of the facial feature.

Another aspect of the present disclosure is directed to a system for assessing or at least partially reconstructing an image of one or more facial regions of a user. The system may comprise: a processor; and a computer-readable medium communicatively coupled to the processor and having non-transitory, processor-executable instructions stored thereon, wherein execution of the instructions causes the processor to perform a method. The method may comprise receiving an input image of at least a portion of a face; using one or more trained machine learning algorithms configured to: segment the input image into one or more regions, identify which of the one or more regions are a region of interest, and classify the regions of interest into one of: a mouth region, a lip region, a teeth region, or a gum region; using a shape predictor algorithm configured to identify a location of the one or more classified regions of interest in the input image; receiving, at a display, a user input selection of a template comprising a desired aesthetic for one or more of the classified regions of interest; applying one or more characteristics of the selected template to the input image; and outputting, to the display, an output image comprising the desired aesthetic of the one or more regions based on the selected template and said applying.

In any of the preceding embodiments, the system further comprises an image sensor communicatively coupled to the processor and configured to take the input image of the at least a portion of the face.

In any of the preceding embodiments, the method performed by the processor further comprises aligning a midpoint of the selected template with a midpoint of the region of interest.

In any of the preceding embodiments, the portion of the face comprises one or more of: a mouth, one or more teeth, a nose, one or both lips, or a combination thereof.

In any of the preceding embodiments, the method performed by the processor further comprises outputting the output image having the desired aesthetic comprises outputting the output image having a desired smile appearance.

In any of the preceding embodiments, the method performed by the processor further comprises providing one or more educational materials related to health of the body region.

In any of the preceding embodiments, the method performed by the processor further comprises: receiving one or more user inputs related to hygiene of the one or more facial regions; ranking the one or more user input based on a health guideline; and generating an oral health score report based on said ranking.

In any of the preceding embodiments, the one or more trained machine learning algorithms comprise a mask R-Convolutional Neural Network architecture using a Residual Network and Feature Pyramid Network backbone.

In any of the preceding embodiments, the shape predictor algorithm is a dlib shape predictor algorithm.

In any of the preceding embodiments, the one or more identified regions comprise: a lip region, individual teeth, a cuspid point, a right corner position of a mouth, a left corner position of the mouth, a mouth coordinate position, a mouth area, or a combination thereof.

In any of the preceding embodiments, applying comprises using the cuspid point as an initial reference to apply the template to a mouth region in the image.

In any of the preceding embodiments, applying further comprises: warping the template, resizing the template for a best fit to the body region in the input image, adjusting one or both of a brightness or a contrast of the template to match with the input image, replacing the template in the body region, or a combination thereof.

In any of the preceding embodiments, the classified region is a gum region such that the method further comprises identifying a gum color of the gum region in the input image and applying a desired gum color to the input image.

In any of the preceding embodiments, the classified region is the mouth region, such that the method further comprises filling one or more corridors of the mouth region with nearest pixel values.

In any of the preceding embodiments, the method performed by the processor further comprises displaying, on the display, one or more guides for positioning of the at least a portion of the face in the input image.

In any of the preceding embodiments, the method performed by the processor further comprises outputting an error message when the one or more features are out of a predetermined range.

In any of the preceding embodiments, the system further comprises the display, such that the processor is communicatively coupled to the display.

In any of the preceding embodiments, the processor is located in a server, remote computing device, or user device.

Another aspect of the present disclosure is directed to a system for customizing a facial feature of an image. The system may comprise: a processor; and a computer-readable medium communicatively coupled to the processor and having non-transitory, processor-executable instructions stored thereon, wherein execution of the instructions causes the processor to perform a method. The method may comprise: receiving an input image having a facial feature identified for customization; identifying a plurality of facial landmark coordinates for the input image; presenting to a user, using a display, a plurality of teeth style templates; receiving a selection of one of the teeth style templates; altering the plurality of coordinates of the selected teeth style template to match the plurality of facial landmark coordinates of the input image; and replacing the teeth region of the input image with the altered teeth style template to provide a customized output image.

In any of the preceding embodiments, the facial feature is one or both of: a lip region and a teeth region..

In any of the preceding embodiments, the plurality of facial landmark coordinates corresponds to one or more of: a lip region, a teeth region, cuspid points, a right corner position of a mouth, a left corner position of the mouth, a mouth coordinate position, a mouth area, a left eye, a right eye, or a combination thereof.

In any of the preceding embodiments, the selected teeth style templates comprise a plurality of coordinates.

In any of the preceding embodiments, replacing the teeth region comprises mapping cuspid point coordinates of the selected teeth style template with the cuspid point coordinates of the input image.

In any of the preceding embodiments, altering the selected teeth style template includes one or more of: warping the selected teeth style template for a best fit to the facial feature of the input image, resizing the selected teeth style template for a best fit to the facial feature of the input image, adjusting one or both of a brightness or a contrast of the selected teeth style template to match a brightness or a contrast of the input image, or a combination thereof.

In any of the preceding embodiments, the method performed by the processor further comprises: analyzing the teeth and gums of the input image, and calculating an oral health score based on one or more of: a presence or absence of dental caries, or gum disease; and providing the oral health score to the user.

In any of the preceding embodiments, the method performed by the processor further comprises providing one or more educational materials related to a health of the facial feature.

In any of the preceding embodiments, the processor is located in a server, remote computing device, or user device.

In any of the preceding embodiments, the image is received by the processor from an image library or database.

In any of the preceding embodiments, the system further comprises the display, such that the processor is communicatively coupled to the display.

The term “about” or “approximately,” when used before a numerical designation or range (e.g., to define a length or pressure), indicates approximations which may vary by ( + ) or ( - ) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A computer-implemented method for assessing or at least partially reconstructing an image of one or more facial regions of a user, comprising:
receiving, at a processor, an input image of at least a portion of a face;
using one or more trained machine learning algorithms configured to:
segment the input image into one or more regions,
identify which of the one or more regions is a region of interest, and
classify the regions of interest into one of: a mouth region, a lip region, a teeth region, or a gum region;
using a shape predictor algorithm configured to identify a location of the one or more classified regions of interest in the input image;
receiving, at a display communicatively coupled to the processor, a user input selection of a template comprising a desired aesthetic for one or more of the classified regions of interest;
applying one or more characteristics of the selected template to the input image; and
outputting an output image comprising the desired aesthetic of the one or more regions based on the selected template and said applying.

2. The method of claim 1, further comprising aligning a midpoint of the selected template with a midpoint of the region of interest.

3. The method of claim 1, wherein the portion of the face comprises one or more of: a mouth, one or more teeth, a nose, one or both lips, or a combination thereof.

4. The method of claim 1, wherein outputting the output image having the desired aesthetic comprises outputting the output image having a desired smile appearance.

5. (canceled)

6. The method of claim 1, further comprising:
receiving one or more user inputs related to hygiene of the one or more facial regions;
ranking the one or more user inputs based on a health guideline; and
generating an oral health score report based on said ranking.

7. The method of claim 1, wherein the one or more trained machine learning algorithms comprise a mask R-Convolutional Neural Network architecture using a Residual Network and Feature Pyramid Network backbone.

8. The method of claim 1, wherein the shape predictor algorithm is a dlib shape predictor algorithm.

9. The method of claim 1, wherein the one or more identified regions comprise: a lip region, individual teeth, a cuspid point, a right corner position of a mouth, a left corner position of the mouth, a mouth coordinate position, a mouth area, or a combination thereof.

10. The method of claim 9, wherein applying comprises using the cuspid point as an initial reference to apply the template to a mouth region in the image.

11. The method of claim 1, wherein applying further comprises: warping the template, resizing the template for a best fit to the portion of the face in the input image, adjusting one or both of a brightness or a contrast of the template to match with the input image, replacing the template in the portion of the face, or a combination thereof.

12. The method of claim 1, wherein the classified region is one or both of:
a gum region such that the method further comprises identifying a gum color of the gum region in the input image and applying a desired gum color to the input image, or
the mouth region, such that the method further comprises filling one or more corridors of the mouth region with nearest pixel values.

13. (canceled)

14. The method of claim 1, further comprising displaying one or more guides for positioning of the at least a portion of the face in the input image.

15. The method of claim 14, further comprising outputting an error message when the one or more classified regions of interest are out of a predetermined range.

16. A computer-implemented application system for assessing and customizing a facial feature of an image, the application comprising:
a user interface having a plurality of interaction screens associated with a processor, the user interface configured to receive user interaction;
an image input screen configured to receive an input image of at least one facial feature;
a selection interaction screen for presenting to the user a number of template variations corresponding to the at least one facial feature, each template variation having a plurality of template coordinates, wherein the selection interaction screen is configured to receive a user selection of one or more of the template variations,
the processor being configured to alter the at least one facial feature of the input image based on the one or more selected template variations, and provide a customized image,
wherein the processor is configured to identify a plurality of input image coordinates to use as reference points for mapping to the plurality of template coordinates of the selected one or more template variations; and
an output image interaction screen configured to present the customized image to the user.

17. The computer-implemented application of claim 16, wherein the processor is configured to identify the plurality of input image coordinates by segmenting the input image into at least one region of interest, identifying boundaries of objects in the input image, and annotating each pixel based on the identified boundary.

18. The computer-implemented application of claim 16, wherein the plurality of input image coordinates is facial landmark points corresponding to one or more of: a lip region, individual teeth, cuspid points, a right corner position of a mouth, a left corner position of the mouth, a mouth coordinate position, a mouth area, a left eye, a right eye, or a combination thereof.

19. The computer-implemented application of claim 16, wherein the input image is provided using one or both of: an input image sensor for taking and uploading the input image of the facial feature of the user or uploaded from an image library.

20. The computer-implemented application of claim 16, wherein the at least one facial feature of the input image comprises one or more of: a mouth, one or more teeth, gums, one or both lips, or a combination thereof.

21. The computer-implemented application of claim 16, wherein the number of template variations comprises one or more of: a number of varying gum shades or a number of varying teeth style templates.

22. The computer-implemented application of claim 21, wherein the selection interaction screen is configured to receive a user selection of one of the number of varying teeth style templates, and wherein the processor is configured to alter the selected teeth style template based on the plurality of coordinates of the selected teeth style template and the corresponding identified plurality of input image coordinates including one or more of: warping the selected teeth style template for a best fit to the facial feature of the input image, resizing the selected teeth style template for a best fit to the facial feature of the input image, adjusting one or both of a brightness or a contrast of the selected teeth style template to match a brightness or a contrast of the input image, or a combination thereof.

23. The computer-implemented application of claim 22, wherein the altered selected teeth style template replaces the at least one facial feature of the input image.

24. The computer-implemented application of claim 20, wherein the processor is further configured to analyze the one or more teeth and gums of the input image, and calculate an oral health score based on one or more of: a presence or absence of dental caries, or gum disease; and provide the oral health score to the user.

25. (canceled)

26. A method for customizing a facial feature of an image, the method comprising:
receiving, at a processor, an input image having a facial feature identified for customization, wherein the facial feature is one or both of: a lip region and a teeth region;
identifying, at the processor, a plurality of facial landmark coordinates for the input image, wherein the plurality of facial landmark coordinates corresponds to one or more of: a lip region, a teeth region, cuspid points, a right corner position of a mouth, a left corner position of the mouth, a mouth coordinate position, a mouth area, a left eye, a right eye, or a combination thereof;
transmitting to a user a plurality of teeth style templates;
receiving, at the processor, a selection of one of the teeth style templates, wherein the selected teeth style templates comprise a plurality of coordinates; and
altering the plurality of coordinates of the selected teeth style template to match the plurality of facial landmark coordinates of the input image; and
replacing the teeth region of the input image with the altered teeth style template to provide a customized output image.

27. The method of claim 26, wherein replacing the teeth region comprises mapping cuspid point coordinates of the selected teeth style template with the cuspid point coordinates of the input image.

28. The method of claim 26, wherein altering the selected teeth style template includes one or more of: warping the selected teeth style template for a best fit to the facial feature of the input image, resizing the selected teeth style template for a best fit to the facial feature of the input image, adjusting one or both of a brightness or a contrast of the selected teeth style template to match a brightness or a contrast of the input image, or a combination thereof.

29. The method of claim 26, further comprising:
analyzing, at the processor, the teeth and gums of the input image, and calculating an oral health score based on one or more of: a presence or absence of dental caries, or gum disease; and
transmitting the oral health score to the user.

30-56. (canceled)
